(19) United States
(12) Patent Application Publication
(10) Pub. No.: US 2023/0335271 A1
(43) Pub. Date: Oct. 19, 2023

(54) Monitoring Vital Signs via Machine Learning

(71) Applicant: VitalTracer Ltd., Cote-Saint-Luc (CA)

(72) Inventors: Azadeh Dastmalchi, Cote St. Luc (CA); Ali Ghorbani, Cote St. Luc (CA)

(73) Assignee: VitalTracer Ltd., Cote-Saint-Luc (CA)

(21) Appl. No.: 17/719,896

(22) Filed: Apr. 13, 2022

(51) Int. Cl. G16H 40/63; G06N 20/00

(52) U.S. Cl. CPC G16H 40/63; G06N 20/00

(57) ABSTRACT

Monitoring vital signs via machine learning is provided. A system can identify data points of signals detected via skin of a user by an optical sensor that indicate changes in volume of blood flowing through a capillary at the skin. The system generates features from the data points. The system inputs the features into a model to produce output. The model can be trained using training data including features and labels corresponding to blood pressure measurements from a reference device. The system determines a value of blood pressure for the user based on the output from the model. The system provides an indication of the value of blood pressure via an interface.

### BACKGROUND

A device can measure a vital sign, such as blood pressure, of a subject. A device to measure a vital sign that is bulky or cuff-based may be uncomfortable for day-to-day use, and not desired. However, devices that are compact may be less accurate when measuring vital signs for certain subjects relative to a larger, more bulky device.

### SUMMARY

Devices can be used to monitor vital signs for patients in hospitals and senior living facilities. Vital signs for such patients can be monitored multiple times a day. These procedures can be time consuming and challenging for disabled patients and patients who suffer from dementia. However, devices used to monitor vital signs can be inefficient, or provide inaccurate or unreliable readings. Systems, methods and apparatus of this technical solution can reliably, accurately and efficiently monitor vital signs, such as blood pressure, in a non-invasive manner without utilizing bulky, time consuming cuff devices.

Described herein are systems, methods and apparatus for monitoring vital signs, such as blood pressure, by using pulse measurements and machine learning techniques. The devices described herein can be wearable devices, such as a smartwatch or a patch, or card devices, and can operate continuously and non-invasively, providing highly accurate vital sign readings without encumbering or agitating patients. Unlike other blood pressure monitoring devices, which utilize mechanical sensors and techniques to convert detected vibrations or sound to an estimate of blood pressure, the techniques described herein utilize optical sensors and machine learning to estimate systolic and diastolic blood pressure accurately. To do so, the techniques described herein can utilize photoplethysmogram (PPG) signals captured using optical sensors, which are subjected to signal processing and input to a machine learning model. The machine learning model outputs an accurate estimate of systolic and diastolic blood pressure.

At least one aspect of the present disclosure can be directed to a system to monitor a vital sign. The system can include a data processing system comprising one or more processors, coupled to memory. The system can identify a plurality of data points of signals detected via skin of a user by an optical sensor that indicate changes in volume of blood flowing through a capillary at the skin. The system can generate a plurality of features from the plurality of data points. The system can input the plurality of features into a model to produce output. The can be model trained using machine learning on training data having values for the plurality of features and labels corresponding to blood pressure measurements from a reference device different from the optical sensor. The system can determine, based on the output from the model, a value of blood pressure for the user. The system can provide an indication of the value of blood pressure via an interface.

In some implementations, the signals are PPG signals obtained from the optical sensor. The system can execute a derivative of the plurality of data points to generate a first feature of the plurality of features. The system can execute a second derivative of the plurality of data points to generate a second feature of the signals. The system can update the value of the blood pressure based on the first feature and the second feature input into the model. A third feature of the plurality of features can include heart rate variability.

The system can pre-process the signals detected by the optical sensor to generate the plurality of data points using at least one of a normalization technique, detrending technique, or a smoothing technique. The system can filter the signals based on a frequency range to generate the plurality of data points. The system can apply a peak detection technique to the signals to generate the plurality of data points.

In some implementations, the system can identify a plurality of peaks in the signals and a plurality of troughs in the signals. The system can generate a plurality of splices of the signals based on the plurality of troughs. The system can discard one or more of the plurality of splices having a duration greater than a threshold. The system can generate the plurality of data points absent the one or more of the plurality of splices discarded responsive to the duration of the one or more of the plurality of splices being greater than the threshold.

The machine learning technique can include a random forest machine learning technique. The system can receive, via a network from a computing device worn by the user, the signals. The computing device can include the optical sensor that detects the signals via the skin of the user. In some implementations, the system can include a computing device worn by the user. In some implementations, the computing device includes the data processing system. The interface can include a display to provide the indication of the value of blood pressure.

In some implementations, the system can receive the training data. The training data can include, for each of a plurality of users: (i) values for the plurality of features generated from a predetermined number of data points of signals corresponding to a predetermined number of heart beats of the plurality of users; and (ii) blood pressure observations measured by the reference device for each of the predetermined number of heart beats, wherein the reference device is different from the optical sensor. A first set of the training data can correspond to the plurality of users performing a first level of physical activity. A second set of the training data can correspond to the plurality of users performing a second level of physical activity different from the first level of physical activity. A third set of the training data can correspond to the plurality of users performing a third level of physical activity that is different from the first level of physical activity and the second level of physical activity.

The system can detect, via the optical sensor, a color of the skin of the user. In some implementations, the system can adjust an intensity of a frequency of light emitted based on the color of the skin to reduce erroneous data points of the plurality of data points.

An aspect of the present disclosure can be directed to a method. The method can be performed, for example, by a data processing system including one or more processors coupled to memory. The method can include identifying a plurality of data points of signals detected via skin of a user by an optical sensor that indicate changes in volume of blood flowing through a capillary at the skin. The method can include generating a plurality of features from the plurality of data points. The method can include inputting the plurality of features into a model to produce output. The model can be trained using machine learning on training data having values for the plurality of features and labels corresponding to measurements of the vital sign from a reference device different from the optical sensor. The method can include determining, based on the output from the model, a value of the vital sign for the user. The method can include providing an indication of the value of the vital sign via an interface.

The signals can be photoplethysmogram (“PPG”) signals obtained from the optical sensor. The method can include executing a derivative of the plurality of data points to generate a first feature of the plurality of features.

An aspect of the present disclosure can be directed to an apparatus wearable by a user to monitor a vital sign. The apparatus can include an optical sensor. The apparatus can include a display. The apparatus can include a data processing system comprising one or more processors, coupled to memory. The apparatus can identify a plurality of data points of signals detected via skin of the user by the optical sensor that indicate changes in volume of blood flowing through a capillary at the skin. The apparatus can generate a plurality of features from the plurality of data points. The apparatus can input the plurality of features into a model to produce output. The model can be trained using machine learning on training data having values for the plurality of features and labels corresponding to blood pressure measurements from a reference device different from the optical sensor. The apparatus can determine, based on the output from the model, a value of blood pressure for the user. The apparatus can provide an indication of the blood pressure via the display.

The apparatus can pre-process the signals detected by the optical sensor to generate the plurality of data points using at least one of a normalization technique, detrending technique, or a smoothing technique.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect can be combined with other aspects. Aspects can be implemented in any convenient form. For example, by appropriate computer programs, which may be carried on appropriate carrier media (computer readable media), which may be tangible carrier media (e.g. disks) or intangible carrier media (e.g. communications signals). Aspects can be implemented using suitable apparatus, which may take the form of programmable computers running computer programs arranged to implement the aspect. As used in the specification and in the claims, the singular form of “a,” “an,” and “the” include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates a block diagram of an example system for monitoring vital signs;

FIGS. 2A, 2B, 2C, 2D, and 2E illustrate example views of an example wearable device that can be utilized by the example system depicted in FIG. 1, or the example methods depicted in FIGS. 6-8;

FIGS. 3A, 3B, and 3C illustrate example views of an example card device that can be utilized by the example system depicted in FIG. 1, or the example methods depicted in FIGS. 6-8;

FIGS. 4A and 4B illustrate example views of an example patch device that can be utilized by the example system depicted in FIG. 1, or the example methods depicted in FIGS. 6-8;

FIGS. 5A and 5B illustrate example graphs showing example data points and features that can be provided or utilized by the example system depicted in FIG. 1, or the example methods depicted in FIGS. 6-8;

FIG. 6 illustrates an example flow diagram of a method for monitoring vital signs;

FIG. 7 illustrates an example method for monitoring vital signs;

FIG. 8 illustrates an example method for generating signals used for monitoring vital signs;

FIG. 9 illustrates the an example computer system that can be employed to implement the systems or components depicted in FIGS. 1-4B, or methods depicted in FIGS. 6-8.

### DETAILED DESCRIPTION

Below are detailed descriptions of various concepts related to, and implementations of, techniques, approaches, methods, apparatuses, and systems for monitoring a vital sign using machine learning. The various concepts introduced above and discussed in greater detail below may be implemented in any of numerous ways, as the described concepts are not limited to any particular manner of implementation. Examples of specific implementations and applications are provided primarily for illustrative purposes.

Accurate monitoring of vital signs, such as blood pressure, is challenging for certain patient populations. Monitoring of vital signs is generally time consuming, and is distressing for disabled patients and patients who suffer from dementia. Using a mechanical cuff-based device to monitor blood pressure can be physically cumbersome and often time consuming to operate. Further, electro-mechanical devices can consume excessive energy or battery capacity, and may have large battery packs. The systems, apparatus and methods of this technical solution can address these and other technical issues by providing techniques to monitor blood pressure and other vital signals without utilizing a mechanical cuff-based device.

For example, the systems, apparatus and methods described herein can utilize sensor readings captured from optical sensors, which provide PPG signals. The PPG signals can be processed and input to a model that is trained, via machine learning, to output systolic and diastolic blood pressure readings for the user. Unlike cumbersome cuff-based devices, the devices described herein can be non-invasive and may be comfortably worn without causing distress in disabled patients or patients suffering with dementia. The devices described herein can be or include wearable devices, such as smartwatches or patches, or card devices, and can operate continuously and non-invasively.

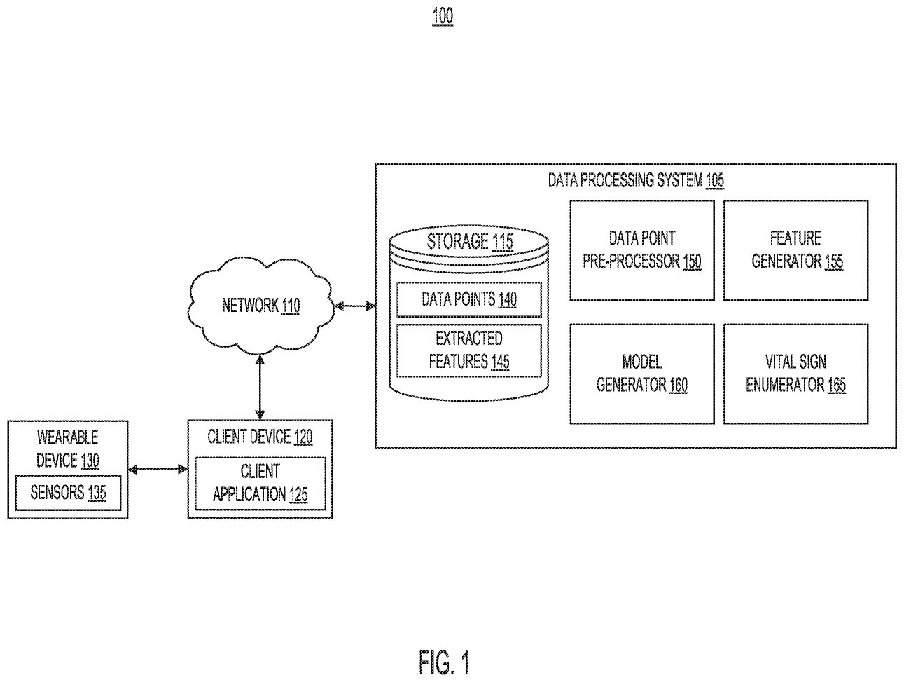

Referring to FIG. 1, illustrated is a block diagram of an example system 100 for monitoring a vital sign, in accordance with one or more implementations. The system 100 can include, utilize, interface with, or otherwise access at least one data processing system 105. The system 100 can include, utilize, interface with, or otherwise access at least one network 110. The system 100 can include, utilize, interface with, or otherwise access at least one client device 120. The system 100 can include, utilize, interface with, or otherwise access at least one wearable device 130. The data processing system 105 can include a storage 115. The data processing system 105 can include at least one data point pre-processor 150. The data processing system 105 can include at least one feature generator 155. The data processing system 105 can include at least one model generator 160. The data processing system 106 can include and at least one vital sign enumerator 165. The storage 115 can include one or more data points 140 and one or more extracted features 145. The client device 120 can execute a client application 125. The wearable device 130 can include one or more sensors 135.

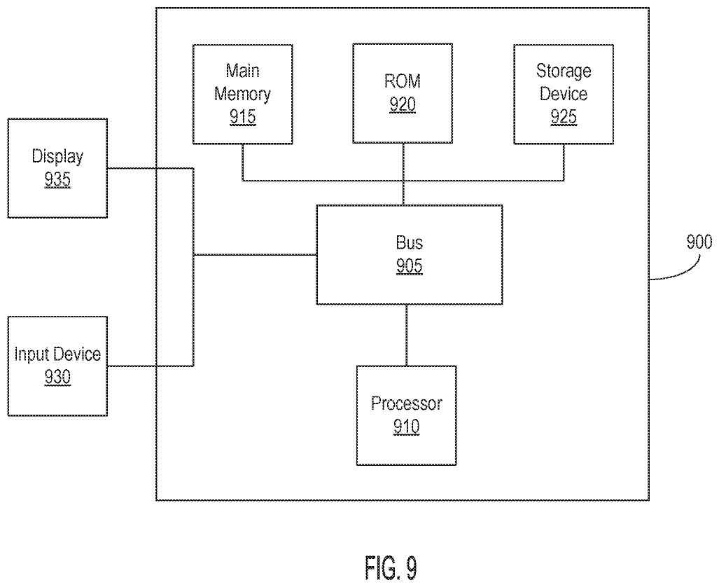

Each of the components (e.g., the data processing system 105, the client device 120, the wearable device 130, the storage 115, the client application 125, the data point pre-processor 150, the feature generator 155, the model generator 160, or the vital sign enumerator 165) of the system 100 can be implemented using the hardware components or a combination of software with the hardware components of a computing system (e.g., computing system 900) depicted in connection with FIG. 9. Each of the components of the data processing system 105, the wearable device 130, or the client device 120 can perform the functionalities detailed herein. Although certain operations or techniques may be described herein from the perspective of the data processing system 105, the client device 120 or the wearable device 130 can perform one or more of those operations or techniques in conjunction or in communication with the data processing system 105 or instead of the data processing system 105. For example, the client device 120 or the wearable device 130 may include the data processing system 105 or one or more components of the data processing system 105.

The data processing system 105 can include at least one processor and a memory (e.g., a processing circuit). The memory (which may include the storage 115) can store processor-executable instructions that, when executed by processor, cause the processor to perform one or more of the operations described herein. The processor may include a microprocessor, an application-specific integrated circuit (ASIC), a field-programmable gate array (FPGA), a system-on-chip (SoC), or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The data processing system 105 can include any or all of the components and perform any or all of the functions of the computer system 900 described herein in connection with FIG. 9. In some implementations, the data processing system 105 may be part of a cloud computing system, and may include one or more computing devices or servers that can perform various functions as described herein.

The client device 120 can include at least one processor and a memory (e.g., a processing circuit). The memory can store processor-executable instructions that, when executed by processor, cause the processor to perform one or more of the operations described herein. The processor may include a microprocessor, an ASIC, an FPGA, an SoC, or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions may include code from any suitable computer programming language. The client device 120 can include any or all of the components and perform any or all of the functions of the computer system 900 described herein in connection with FIG. 9.

The wearable device 130 can include at least one processor and a memory (e.g., a processing circuit). The memory can store processor-executable instructions that, when executed by processor, cause the processor to perform one or more of the operations described herein. The processor may include a microprocessor, a microcontroller, an ASIC, an FPGA, an SoC, or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions may include code from any suitable computer programming language. The wearable device 130 may include any or all of the components and perform any or all of the functions of the computer system 900 described herein in connection with FIG. 9.

The network 110 can include computer networks such as the Internet, local, wide, metro or other area networks, intranets, satellite networks, other computer networks such as voice or data mobile phone communication networks, and combinations thereof. The data processing system 105 of the system 100 can communicate via the network 110, for instance with at least one client device 120. The network 110 may be any form of computer network that can relay information between any of the computing devices described herein. In some implementations, the network 110 may include the Internet and/or other types of data networks, such as a local area network (LAN), a wide area network (WAN), a cellular network, a satellite network, or other types of data networks. The network 110 can include any number of computing devices (e.g., computers, servers, routers, network switches, etc.) that are configured to receive and/or transmit data within the network 110. The network 110 may further include any number of hardwired and/or wireless connections. Any or all of the computing devices described herein (e.g., the data processing system 105, the client device 120, the wearable device 130, the computer system 900, etc.) may communicate wirelessly (e.g., via WiFi, Bluetooth, cellular, radio, etc.) with a transceiver that is hardwired (e.g., via a fiber optic cable, a CATS cable, etc.) to other computing devices in the network 110. Any or all of the computing devices described herein (e.g., the data processing system 105, the client device 120, the wearable device 130, the computer system 900, etc.) can communicate wirelessly with the computing devices of the network 110 via a proxy device (e.g., a router, network switch, or gateway).

The client device 120 can include, but is not limited to, a personal computer, a laptop computer, a smart phone device, a mobile device, or another type of computing device. Each client device 120 can be implemented using hardware or a combination of software and hardware. Each client device 120 can include a display device, such as a liquid crystal display (LCD), a light-emitting diode (LED) display, or an organic light-emitting diode (OLED) display, among others. The display may be an interactive display such as a touchscreen. The client device 120 can include one or more input devices (e.g., a mouse, a keyboard, digital key pad, touchscreen, etc.) that can be used to receive user input. The display can be used to present information from the

The client device 120 can execute a client application 125. The client application 125 can display information relating to sensor data captured by the wearable device 130. The client application 125 may present one or more user interfaces to a user, which may include various interactive user interface elements that allow a user to connect to a wearable device 130, request sensor data from the wearable device 130, or otherwise communicate with or configure the wearable device 130. The information displayed by the client application 125 may include any of the data points 140, the extracted features 145, patient information, graphs of sensor data, or other information relating to the information described herein. The client application 125 may include one or more login interfaces or other interfaces that allow a user to upload data points 140 captured by the wearable device 130 or extracted features 145 processed by the client application 125 (or by the data processing system 105). In some implementations, the client application 125 can perform any or all of the functionality of the data processing system 105.

The client device 120 can include one or more communications interfaces, with which the client device 120 (or the client application 125) can utilize to communicate information with other computing devices, such as the data processing system 105 or the wearable device 130. The communications interfaces can include one or more wireless communications interfaces (e.g., a wireless-fidelity (Wi-Fi) communication interface, a Bluetooth communications interface, a Zigbee communications interface, a near-field communications (NFC) interface, etc.) or one or more wired communications interfaces (e.g., an Ethernet interface, a serial interface such as a universal serial bus (USB), a parallel interface, a discrete signal interface, etc.). The client device 120 can utilize the communications interfaces to retrieve one or more data points 140 captured by the sensors of the wearable device 130. The retrieved data points 140 may be stored in one or more data structures in the memory of the client device, and can be transmitted via the network 110 to the data processing system 105. In some implementations, the client application 125 executing on the client device 120 can perform one or more pre-processing techniques, as described herein. Additionally, the client application 125 may perform any of the functionality of the data processing system 105, or any of its components, including the data point pre-processor 150, the feature generator 155, the model generator 160, and the vital sign enumerator 165. Likewise, in some implementations, the storage 115 (and any data structures stored therein) may be part of, or otherwise in communication with or accessible by, the client device 120.

The wearable device 130 may be any type of device that can be placed on, in contact with, proximate to, or fixed to the skin of a user. The wearable device 130 can contact the skin of the user or subject. In some cases, the wearable device 130 can be located within a proximity or distance of the skin of the user such that PPG signals can be detected by an optical sensor 135 of the wearable device 130. For example, the optical sensor 135 can detect signals reflected from or emitted via the skin of the user while the wearable device 130, or one or more components thereof, are not be in contact with or affixed to the skin of the user.

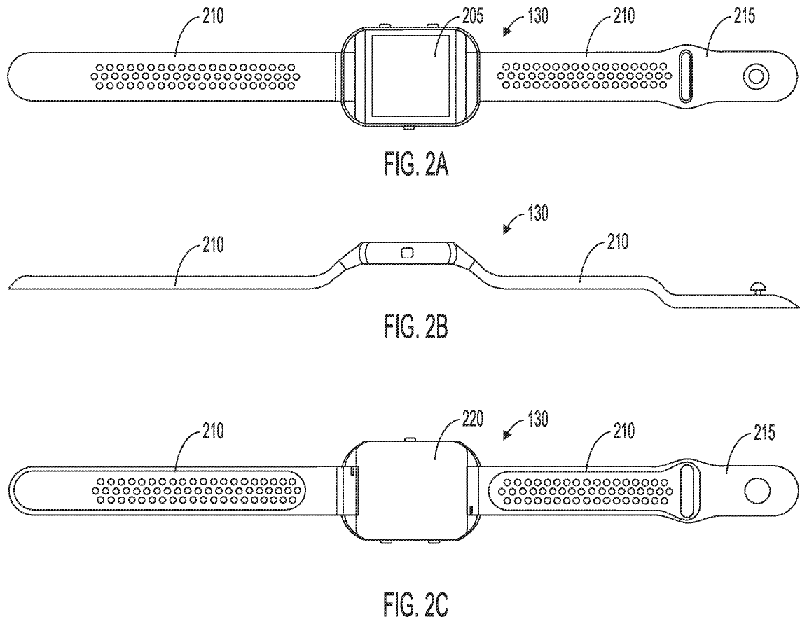

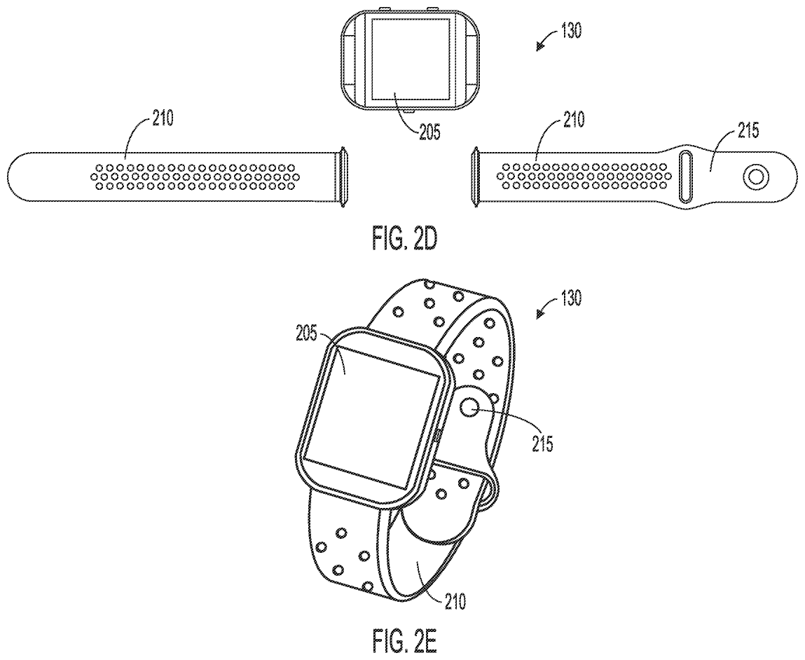

The wearable device 130 can be or include a smart watch, a patch, a card, or may make up a portion of clothing of the user. Example implementations of the wearable device are shown and described in connection with FIGS. 2A-2E, 3A-3C, and 4A and 4B. The wearable device 130 can include one or more communications interfaces, which may be utilized to communication with one or more of the computing devices described herein. For example, the wearable device 130 may utilize the communications interfaces to communication one or more data points 140 to the client device 120, or in some implementations may communication data points to the data processing system 105 via the network 110. The communications interfaces may include one or more wireless communications interfaces (e.g., a Wi-Fi communication interface, a Bluetooth communications interface, a Zigbee communications interface, an NFC interface, etc.) or one or more wired communications interfaces (e.g., an Ethernet interface, a serial interface such as a USB, a parallel interface, a discrete signal interface, etc.).

The wearable device 130 may include one or more user interface devices, including input devices, such as buttons or actuators that receive user input, and output devices, such as one or more displays or audio-producing devices (e.g., speakers, alarms, etc.). The wearable device 130 can include a display that may present any of the information captured by the sensors 135 of the wearable device 130, including PPG information, heartrate information, or other sensor data described herein. The display can be any type of display, including an LCD display, an LED display, an OLED display, or a bi-stable display (e.g., e-ink, etc.), among others. The wearable device 130 may include a portable power source, such as a battery (e.g., a lithium ion, alkaline, nickel metal hydride, etc.) or a device that can generate electric energy, such as a solar cell.

The wearable device 130 can include one or more sensors 135. The sensors 135 can capture sensor data (e.g., the data points 140), which may be stored in the memory of the wearable device 130, and subsequently transmitted to another computing device, such as the client device 120 or the data processing system 105. The sensors 135 can include various sensors that capture information relating to vital signals of the user. The sensors 135 can include optical sensors (e.g., which may provide PPG readings), ECG sensors, UV sensors, motion sensors, ambient temperature sensors, humidity sensors, pressure sensors, skin temperature sensors, and blood sugar sensors, among others. Data points 140 captured from the sensors 135 may be stored in the memory of the wearable device 130 in association with a timestamp indicating when each data point 140 is captured. In some implementations, the wearable device 130 can initiate capturing of data points 140 from the sensors 135 in response to a request. The request may be provided via input to an input device of the wearable device, via a request received from the client device 120, or via a request received from the data processing system 105.

The request can identify particular sensors 135 from which data points 140 are to be captured, and may indicate additional metadata. The additional metadata can indicate an amount of time that the sensors 135 should capture data points 140, various configuration settings for the sensors 135 (e.g., calibration information, configuration information such as data resolution, etc.). In response to the request, the wearable device 130 can capture the data points 140 from the identified sensors 135 (or from a default set of sensors 135) in accordance with the metadata, if present. The wearable device 130 can transmit any captured data points 140 to the client device 120 or the data processing system 105 in response to a request for sensor data.

The wearable device 130 can perform one or more operations to optimize capturing of data points 140 that are useful for the techniques described herein. For example, the wearable device 130 can detect a color of the skin of the user via an optical sensor of the sensors 135. To do so, the wearable device 130 may capture one or more optical sensor readings. For example, the wearable device 130 can transmit one or more optical signals (e.g., of a predetermined intensity) using a light source of the sensors 135, and capture the reflected light using the optical sensor. The reading indicated by the optical sensor may be, for example, an intensity value. The wearable device 130 can compare the retrieved optical sensor intensity value to one or more stored reference values (e.g., captured using light emitted at the same predetermined intensity) that each correspond to a respective skin color. In some implementations, the wearable device 130 may transmit the intensity values to the client device 120 or the data processing system 105, which can then perform the comparison. The wearable device 130 (or the client device 120 or the data processing system 105) can estimate the skin color of the user based on the comparison (e.g., the reference value to which the sensor readings most closely match can indicate the corresponding skin color).

Based on the skin color, the wearable device 130 can adjust an intensity of a frequency of light emitted based on the color of the skin to reduce erroneous data points of the plurality of data points. For example, when an optical sensor captures PPG signals using an optical sensor at certain light frequency levels, certain skin colors may not provide accurate or useful data points 140 for the techniques described herein. To compensate for these differences, the wearable device 130 can adjust a frequency of the light emitted by the optical sensor. The different frequencies of light for respective skin colors can be stored in a lookup table. The lookup table can be established by an administrator or operator of the data processing system 105. In some cases, the lookup table can be established, generated, or updated using a model training via machine learning. For example, the model generator 160 can generate the lookup table based on output from the model or performance feedback. Once the skin color of the user has been determined, the wearable device 130 can retrieve the corresponding frequency that is optimized for that skin color from the lookup table, and utilize that frequency with the optical sensor to capture the PPG data points 140 as described herein. To utilize the frequency, the wearable device 130 may instruct the optical sensor with a configuration setting that corresponds to the desired frequency. In some implementations, the client device 120 or the data processing system 105 can determine the skin color and the frequency, and provide the desired frequency as part of the metadata in a request for sensor data.

The storage 115 can be a computer memory configured to store or maintain any of the information described herein. The storage 115 can store one or more data structures, which may contain, index, or otherwise store each of the values, pluralities, sets, variables, vectors, or thresholds described herein. The storage 115 can be accessed using one or more memory addresses, index values, or identifiers of any item, structure, or region maintained in the storage 115. The storage 115 can be accessed by the components of the data processing system 105, or any other computing device described herein via the network 110. The storage 115 may be internal to the data processing system 105. In some implementations, the storage 115 can exist external to the data processing system 105, and may be accessed via the network 110. The storage 115 may be distributed across many different computer systems or storage elements, and can be accessed via the network 110 or a suitable computer bus interface. The data processing system 105 can store, in one or more regions of the memory of the data processing system 105, or in the storage 115, the results of any or all computations, determinations, selections, identifications, generations, constructions, or calculations in one or more data structures indexed or identified with appropriate values. Any or all values stored in the storage 115 may be accessed by any computing device described herein, such as the data processing system 105, to perform any of the functionalities or functions described herein.

The storage 115 can store one or more data points 140, for example, in one or more data structures. The data points 140 can include individual or aggregate readings from the sensors 135 of one or more wearable devices. The data structures that store the data points 140 may include a stored association with an identifier of a patient from which the data points 140 were captured. The data points 140 can be associated in the storage 115 with one or more timestamps corresponding to the time that the data points 140 were captured by the sensors 135. The data points 140 may be time series data points 140, which include time series readings from each of the sensors 135 at the wearable device 130. The data points 140 can include readings from any type of sensor, including optical sensors, electrocardiogram (ECG) sensors, ultraviolet (UV) sensors, motion sensors, temperature sensors, humidity sensors, pressure sensors, and blood sugar sensors, among others. The data points 140 may be processed to determine one or more vital signs for a patient.

The storage 115 can store one or more extracted features 145, for example, in one or more data structures. The extracted features 145 may be features that are extracted by processing one or more of the data points 140 using the techniques described herein. The extracted features 145 can include derivative values of the data points 140. For example, using the techniques described herein, the data processing system 105 can calculate the first, second, third, and fourth derivative values of the data points 140 corresponding to PPG values. The extracted features 145 can include frequency-domain specific features, such as a fast Fourier transform (FFT) of one or more sets of the data points 140, or heart rate variability values generated from one or more sets of the data points 140. In some implementations, the extracted features 145 can be generated from data points 140 that are pre-processed using various techniques described herein. For example, the extracted features 145 can be generated from sequences of data points 140 corresponding to valid cycles of PPG readings, as described in greater detail herein. The extracted features 145 can be utilized as input data for the model generator 160, which can generate output data indicating blood pressure values or other vital signs. The extracted features 145 can also include other information calculated from the data points 140, such as mean-time domain features and composite features.

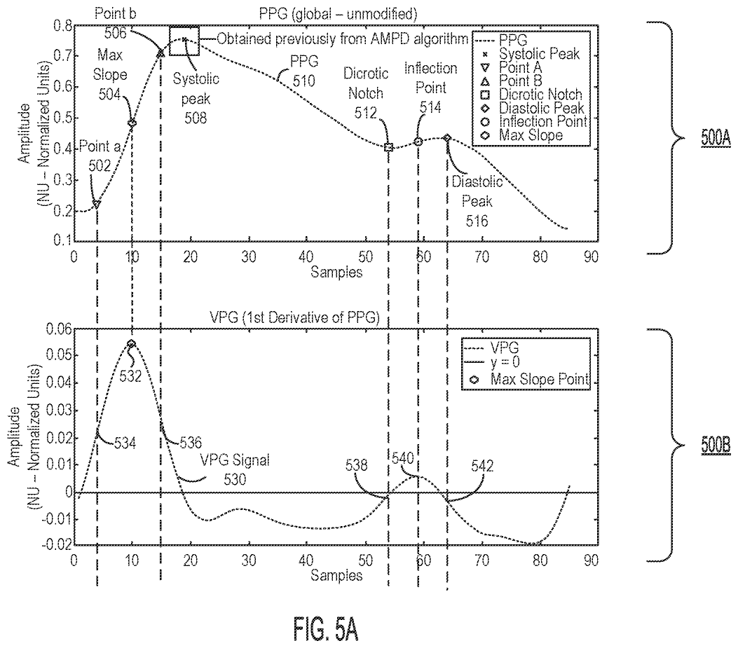

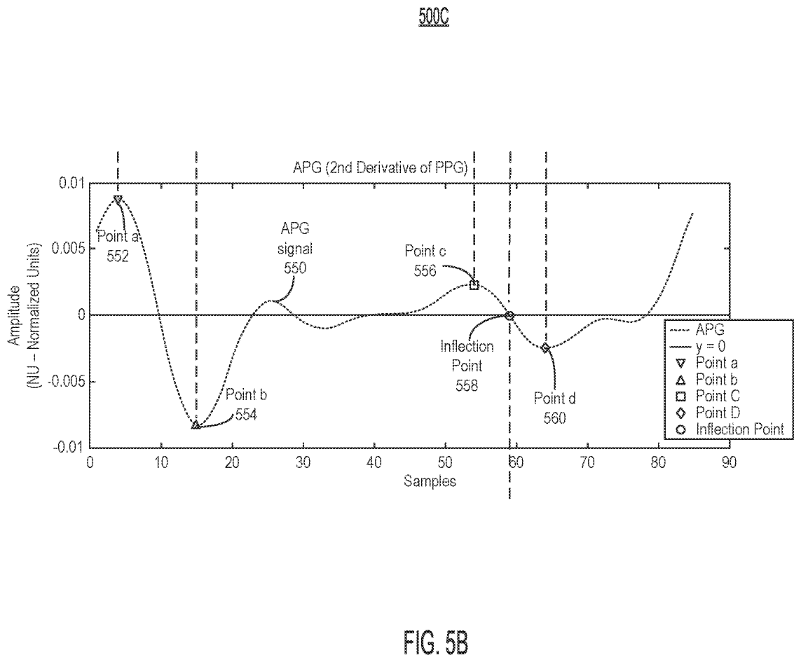

Referring to FIGS. 5A and 5B, and others, illustrated are example graphs 500A, 500B, and 500C showing example data points 140 and extracted features 145 that may be utilized with the techniques described herein. As shown in the graph 500A of FIG. 5A, the top portion of the graph indicates one or more data points 140, which in this implementation correspond to PPG readings captured by an optical sensor. The PPG data making up the displayed data points 140 may be pre-processed or raw data. The units of the x-axis of the graph 500A can be samples, and the units of the y-axis of graph 500A can be amplitude. The x-axis can include approximately 85 samples. The number of samples for a PPG signal can correspond to consecutive troughs or peaks of the PPG signal 510, for example. The amplitude of the PPG signal can be normalized from 0 to 1, for example.

The graph 500A can indicate one or more data points or samples of interest of a PPG signal 510 that can be used to generate a feature for input into a machine learning model. The graph 500A of the PPG signal 510 can include one or more points, such as point a 502 and point b 506. The graph 500A can indicate an amplitude and sample of the PPG signal 510 that corresponds to an instantaneous maximum slope 504. The graph 500A can indicate an amplitude and sample the PPG signal 510 that corresponds to a systolic peak 508. The graph 500A can indicate an amplitude and sample of the PPG signal 510 that corresponds to a dicrotic notch 512. The graph 500A can indicate an amplitude and sample of the PPG signal 510 that corresponds to an inflection point 514. The graph 500A can indicate an amplitude and sample of the PPG signal 510 that corresponds to a diastolic peak 516. The data points 502, 504, 506, 508, 512, 514, or 516 can be features (e.g., extracted feature 145) of the PPG signal 510 that can be input into a machine learning model to determine a value of a vital sign, or used to train a machine learning model to determine values of a vital sign.

Graph 500B can represent a VPG signal 530. The VPG signal 530 can be a first derivative of the PPG signal 510 depicted in graph 500A. The units of the x-axis of graph 500B can be samples. The x-axis samples depicted in graph 500B can be aligned or correspond to the samples of the PPG signal 510 depicted in graph 500A. The y-axis of the graph 500B can be amplitude. The graph 500B can depict points or samples that correspond to samples or points of interest in graph 500A. The graph 500B can indicate features of a VPG signal 530 that can be input into a machine learning model to determine a vital sign.

For example, data point 532 of graph 500B has an amplitude and sample number of the VPG signal 530 that corresponds to a maximum slope point 504 of the PPG signal 510. Data point 534 of VPG signal 530 can correspond to point a 502 of PPG signal 510. Data point 536 of VPG signal 530 can correspond to point b 506 of PPG signal 510. Data point 538 of VPG signal 530 can correspond to the dicrotic notch 512 of PPG signal 510. Data point 540 of VPG signal 530 can correspond to the inflection point 514 of PPG signal 510. Data point 542 of VPG signal 530 can correspond to the diastolic peak 516 of PPG signal 510. The data points 534, 532, 536, 538, 540 or 542 can be features (e.g., extracted features 145) of the VPG signal 530 that can be input into a machine learning model to determine a value of a vital sign, or used to train a machine learning model to determine values of a vital sign.

FIG. 5B depicts a graph 500C illustrating an APG signal 550. The APG signal can be a derivative of the VPG signal 530 depicted in graph 500B, or a second derivative of the PPG signal 510 depicted in graph 500A. The graph 500C can include one or more data points corresponding to points of interest or features depicted in graph 500A or 500B. For example, data point a 552 can correspond to data point a 534 of VPG signal 530 and data point a 502 of PPG signal 510. Data point b 554 of APG signal 550 can correspond to data point b 536 of VPG signal 510, which can correspond to data point b of PPG signal 510. Data point c 556 of APG signal 550 can correspond to data point 538 of VPG signal 530, which can correspond to the dicrotic notch 512 of PPG signal 510. Inflection data point 558 of APG signal 550 can correspond to data point 540 of VPG signal 530, which can correspond to the inflection point 514 of PPG signal 510. Data point d 560 of APG signal 550 can correspond to data point 542 of VPG signal 530, which can correspond to the diastolic peak 516 of PPG signal 510. The data points 552, 554, 556, 558 or 560 can be features (e.g., extracted features 145) of the APG signal 550 that can be input into a machine learning model to determine a value of a vital sign, or used to train a machine learning model to determine values of a vital sign.

Referring again to FIG. 1, and to the operations of the data processing system 105, the data point pre-processor 150 can identify one or more data points 140 of signals detected via skin of a user by one or more of the sensors 135. For example, the data points 140 may include PPG readings captured by an optical sensor of the wearable device 130. When represented as a time series, the data points 140 can indicate changes in volume of blood flowing through a capillary at the skin of the user. The data points 140 may be identified, for example, by transmitting a request for data points to the client device 120 or to the wearable device 130. In response to the request, the client device 120 or the wearable device 130 can transmit the raw data points 140 to the data processing system 105, which may be time series data corresponding to a time period that the raw data points 140 were captured. The data processing system 105 can store the raw data points 140 in association with an identifier of the patient from which the data points 140 were captured, and in association with various metadata indicating, for example, the time period corresponding to the raw data points 140, the skin color of the user from which the data points 140 were captured, or an identifier of the wearable device 130 from which the raw data points 140 were captured.

The data point pre-processor 150 can perform pre-processing on the raw data points 140 captured by the sensors 135 of the wearable device 130. For example, the pre-processor 150 can process the data points prior to the data processing system 105 performing one or more feature extraction processes. Pre-processing the raw data points 140 can include performing at least one of a normalization technique, a detrending technique, or a smoothing technique. At a first stage of the pre-processing process, the data point pre-processor 150 can normalize the raw data points to the range from zero (0) to one (1), inclusive. The data point pre-processor 150 can apply a detrending technique, which may include using a best straight-fit line from the normalized data points 140 for a detrend function with default parameters. The data point pre-processor 150 can apply a bandpass filter to the detrended data points 140. The bandpass filter may be any suitable type of bandpass filter, such as Chebyshev type II 4^{th} order bandpass filter. The bandpass filter may filter the detrended data points 140 to a predetermined frequency range, such as 0.5 Hz to 10 Hz, and a predetermined attenuation, such as 20 db.

After applying the bandpass filter, the data point pre-processor 150 can apply further pre-processing operations. For example, the data point pre-processor 150 may apply a smoothing function. The smoothing function may be a three-point multi-pass moving average filter. The smoothing function may be applied for a predetermined number of iterations (e.g., three iterations). After applying the smoothing technique, the data point pre-processor 150 can then normalize the data points to the range from zero (0) to one (1), inclusive. Performing these pre-processing steps on the raw data points 140 corresponding to PPG signals can accentuate different properties of interest in the PPG signals, such as the systolic peaks, diastolic peaks, and dicrotic notches (e.g., denoting a pulse in which a double beat is detectable for each beat of the heart).

After performing the aforementioned pre-processing techniques, the data point pre-processor 150 can perform a peak detection process to identify peaks in the pre-processed data points 140. These detected peaks can be used to extract segments of the data points 140 that correspond to heart beats of the user. The heartbeat segments can then be used in a feature extraction process, as described in greater detail herein. The process to identify segments of the pre-processed data points 140 that correspond to heartbeats is sometimes referred to herein as a signal quality index (SQI) function. At the start of the SQI function, the data point pre-processor 150 can apply a peak detection function to the pre-processed data points 140 to identify one or more systolic peaks. The peak detection function may be, for example, an automatic multiscale-based peak detection (AMPD) function. The peak detection function may be executed a predetermined number of times over the pre-processed data points 140 (e.g., two passes, etc.). For example, a first pass of the AMPD function can be used to detect each peak in the pre-processed data points 140, and a second pass of the AMPD function can be used to detect each trough in the pre-processed data points 140.

Once the peaks and troughs have been identified in the pre-processed data points 140, the data point pre-processor 150 can generate one or more splices, or segments, of the pre-processed data points 140. These segments can be generated by splicing the pre-processed data points 140 at each of the troughs. Once the segments are generated, the data point pre-processor 150 can calculate a time interval for each of the segments (e.g., by subtracting the timestamp of the first data point 140 in the segment from the timestamp of the last data point 140 in the segment). The data point pre-processor 150 can then calculate the mean time interval of all segments by summing each of the segment time intervals and dividing the sum by the number of segments.

The data point pre-processor 150 can use the mean time interval for the segments to determine which of the segments (which correspond to heartbeats) are valid. The valid segments can be utilized in further operations described herein, while the invalid segments can be discarded. To determine which of the segments are valid, the data point pre-processor 150 can compare the time interval of each of the segments to the mean time interval. Any segments having a time interval that is more than 1.5 times greater than the mean segment time interval can be considered invalid, and discarded. Additionally, the data point pre-processor 150 can discard any segments of the pre-processed data points 140 that do not include a single detected peak between two detected troughs. After discarding the invalid segments, the data point pre-processor 150 is left with a set of valid segments of the pre-processed data points 140, which can be utilized in a feature extraction process.

The feature generator 155 can generate one or more extracted features 145 from the data points 140 of each valid segment. The extracted features 145 can include, for example, derivative values of the PPG signals in the valid segments. The derivative values can include first, second, third, and fourth order derivative values. For example, a first derivate of the PPG can be referred to as a VPG signal; a second derivate of the PPG signal can be referred to as an APG signal; a third derivate of the PPG signal can be referred to as a PPG3 signal; a fourth derivate of the PPG signal can be referred to as a PPG4 signal. The extracted features 145 can include heart rate variability, which can be calculated for each of the valid segments of the data points 140. To calculate a derivative, the feature generator 155 can execute one or more discrete derivative functions, which can take the data points 140 of a segment as input, and produce a corresponding set of derivative data points as output. The process may be repeated by utilizing the set of derivative data points as input to calculate a second derivative, and so on to calculate each of the first, second, third, and fourth derivative values. Each of these extracted features 145 can be stored in association with the respective valid segment of the data points 140. In some implementations, the valid segment of the data points 140 may itself be considered an extracted feature 145. The feature generator 155 can calculate extracted features 145 in the frequency domain, such as FFT each valid segment of data points 140. The feature generator 155 can also calculate the heart rate variability for each of the valid segments as an extracted feature, as well as the mean heart rate variability for all of the valid segments. Once the extracted features 145 have been generated for each valid segment of the data points 140, the extracted features 145 can be provided as input to a trained model, which generates output values corresponding to vital signs of the user.

The model generator 160 can input the extracted features 145 for each valid segment into the trained model to produce one or more output values. The output values can be estimates for the systolic and diastolic blood pressure of the user. The trained model can be a machine-learning model, such as an ensemble random forest model, and may be trained using any suitable supervised, unsupervised, or semi-supervised training model. The model can be trained prior to utilizing the model to estimate vital signs, by using sets of training data including ground truth data (e.g., labels). In some implementations, other types of models can be utilized, including linear regression models, sparse vector machine (SVM) models, neural networks, or other types of regression models.

For example, the model generator 160 can train the model using training data that includes sets of extracted features 145 corresponding to the features extracted by the feature extractor 155, and also includes known values for the systolic and diastolic blood pressure. The sets of training data may correspond to a predetermined number of heartbeats monitored from a number of test subjects. At least three sets of data points 140 can be gathered from each test subject under different conditions. A first set of the training data may gathered from test subjects in a resting, sitting position. A second set of the training data can be gathered from the test subjects in after performing a physical activity, such as jumping jacks. A third set of training data can be gathered from the test subjects after lying down. Each of the three sets of training data can be gathered a predetermined number of times for each test subject. For example, if the sets of training data are gathered for a test subject three times, then nine sets of training data are generated for that test subject.

To gather a set of training data from a test subject, the wearable device 130 can be used to generate raw data points 140 corresponding to a predetermined time period (and therefore, a predetermined number of heartbeats). At that time, systolic and diastolic blood pressure can be monitored and recorded for each heart beat using a reference device, such as a mechanical cuff device. The raw data points 140 for each beat can be provided to the data processing system 105 and pre-processed and segmented, as described herein. After generating the segments, the feature extractor 155 can be used to generate the extracted features 145 for each segment, as described herein. After generating the extracted features 145 for each heartbeat, the known blood pressure values (e.g., the systolic and diastolic blood pressure) for each heartbeat can be stored in association with the corresponding set of extracted features 145, as the ground truth value. This generates one item of training data, which the model generator 160 can use to train the model. The process can be repeated for each heartbeat in each set of training data, to generate sets of training data for a test subject. In some implementations, the training data can be generated for several subjects, and the training data for the several subjects can be used to train a collective model.

To train the model, the model generator 160 can perform a machine learning function using the training data to adjust the parameters of the model. The machine learning function may be a random forest training function, which utilizes the known blood pressure values in each item of training data to compensate for the errors in the model. The trained model can be a regression model, which is trained using the machine learning function to output systolic and diastolic blood pressure values in response to receiving extracted features 145 as input. The machine learning model may be an iterative training function, which trains the model using randomly selected batches of the training data until a predetermined accuracy level is reached.

To determine the accuracy of the model, a subset of the training data can be utilized as a test data set. The test data set can be a set of the training data that is not used to train the model, and is instead only used to evaluate the model accuracy. The accuracy can be evaluated by providing the extracted features 145 in each item of the test data set as input to the model, and comparing the output of the model to the known values in the respective items of test data. The percentage of the output blood pressure values generated by the model that match the respective known blood pressure values in the test data (e.g., with a predetermined tolerance range) can be the overall accuracy of the model. The model generator 160 can repeatedly evaluate the accuracy of the model after training the model with a predetermined amount of training data. Once a predetermined accuracy value is reached, the model generator 160 can store the model in one or more data structures in the storage 115. The model can then be used to predict the blood pressure values (or values for other vital signals, if trained to do so) by using the extracted features 145.

In some implementations, the model generator 160 can generate and train a respective model for each user. For example, sets of training data can be gathered from a user, and a respective model can be trained for that user and later used to predict the blood pressure values (or other vital sign values) for that user. Likewise, in some implementations, a general model may be trained using training data from many users, and the general can be used to predict the blood pressure values (or other vital sign values) for many users, including users that did not necessarily contribute data for training. Once the model has been trained using the training data, the model generator 160 can provide the extracted features 145 as input to the model, and execute the model to produce one or more output values.

The vital sign enumerator 165 can determine a value of blood pressure for the user based on the output from the model. For example, the model may output the systolic and diastolic blood pressure for the user for a given set of input features 145. The vital sign enumerator 165 can store these values in association with an identifier of the user, indicating that the values represent the blood pressure of the user. The vital sign enumerator 165 can store the output values in association with the respective extracted features 145 provided as input to the model. The estimated blood pressure values can be determined based on an average of the blood pressure values generated by the model. For example, the prior operations described herein may generate several valid segments of data points 140 that each represent a heartbeat, and sets of extracted features 145 that correspond to each valid segment. The model can estimate blood pressure values for each set of features 145 spanning a predetermined time window or a predetermined number of heartbeats, and average the values to determine a final blood pressure estimate. In some implementations, the vital sign enumerator 165 can calculate a rolling average.

The vital sign enumerator 165 can provide an indication of the value of blood pressure via an interface. For example, the vital sign enumerator 165 can display the blood pressure values on a display of the data processing system 105. The vital sign enumerator 165 can provide the blood pressure values for display on another computing device, such as the client device 120 or the wearable device 130. For example, the data processing system 105 may transmit the calculated blood pressure values to the client device 120 as they are calculated, such that they can be displayed to the user via the client application 125. The client application 125 can transmit the blood pressure values to the wearable device 130, which can be presented on a display device of the wearable device 130.

Referring to FIGS. 2A, 2B, 2C, 2D, and 2E, and others, illustrated are example views of an example wearable device (e.g., the wearable device 130 described in connection with FIG. 1). FIGS. 2A, 2B, and 2C show top, side, and bottom views, respectively, of an example wearable device 130. As shown, the wearable device 130 may include a display 205. The display 205 can present any of the information captured by the sensors of the wearable device 130, including PPG information, heartrate information, or other sensor data described herein. The display 205 can be any type of display, including an LCD display, an LED display, an OLED display, or a bi-stable display (e.g., e-ink, etc.), among others. The wearable device 130 may include a portable power source, such as a battery (e.g., a lithium ion, alkaline, nickel metal hydride, etc.) or a device that can generate electric energy, such as a solar cell.

As shown in FIGS. 2A-2E, the wearable device 130 can be a watch, which includes a housing 220 that houses each of the components of the wearable device 130, such as the display 205, the sensors 135 described in connection with FIG. 1, and any portable power devices. The wearable device 130 shown in FIGS. 2A-2E can include a strap 210, which can be used to couple the wearable device 130 to the skin of a user. The wearable device 130 can include a connector 215, which can be used to fix the strap in place, allowing the sensors of the wearable device 130 to firmly contact the skin of the wearer. In this implementation of the wearable device 130, one or more sensors can be positioned at the bottom of the housing 220 shown in FIG. 2C. The wearable device 130 may include one or more buttons or interactive devices, which allow the user to provide input to the device to perform one or more actions (e.g., to initiate or stop recording of sensor data, to initiate transfer of sensor data to another computing device, etc.).

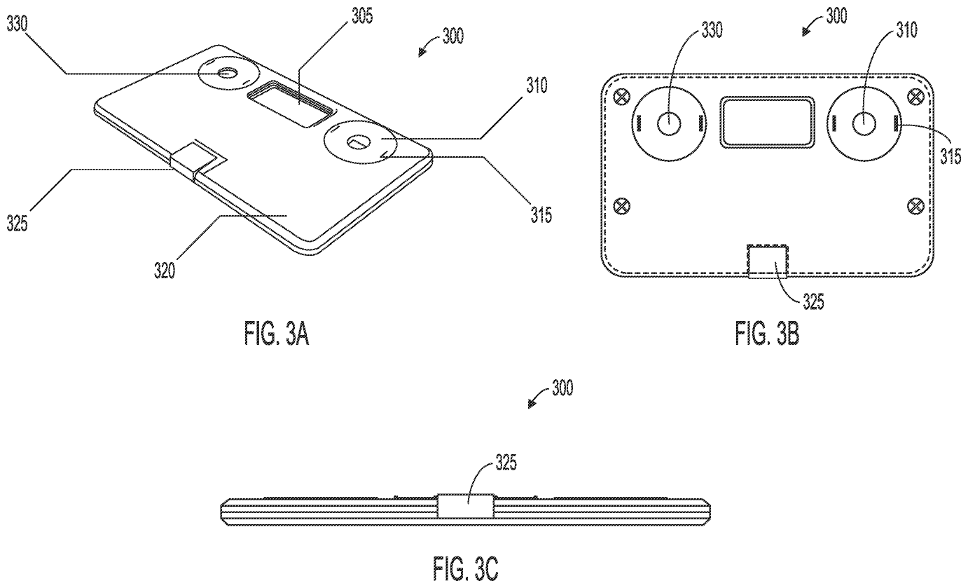

Referring to FIGS. 3A, 3B, and 3C, and others, illustrated are perspective, top, and side views, respectively, of an example card device 300 (e.g., an implementation of the wearable device 130) that may be utilized with the techniques described in connection with FIG. 1. As shown, the card device 300 can include a display 305. The display 305 can be similar to the display 205 described in connection with FIG. 2, and can be or include an LCD display, an LED display, an OLED display, or a bi-stable display (e.g., e-ink, etc.), among others. The card device 300 can include a portable power source, such as a battery (e.g., a lithium ion, alkaline, nickel metal hydride, etc.) or a device that can generate electric energy, such as a solar cell.

The card device 300 can include a number of sensors (e.g., the sensors 135 described in connection with FIG. 1). The sensors can include a PPG sensor 310, which may utilize red, infrared, or green light. The PPG sensor 310 may be surrounded by an ECG sensor, which together with the second ECG sensor 330 can capture ECG signals from a user. The second ECG sensor 330 may include an ultraviolet sensor. The sensors can include motion sensors 320, which can include one or more accelerometers, gyroscopes, magnetometers, or general IMUs. Additionally, the card device 300 can include a glucose strip connector 325, which can be used to gather blood sugar levels from a glucose strip. The signals captured by the sensors of the card device 300 may be communicated to other, external computing devices (not pictured) utilizing one or more communication interfaces, as described in further detail in connection with FIG. 1. To operate the card device 300, the user can press their fingers or thumbs against the left and right pads including the ECG sensors 330 and the PPG sensors 310, such that one or more of the sensors of the card device 300 contact the skin of the user. Readings from the sensors can be presented to the user on the display 305. The user can utilize a glucose strip inserted into the glucose strip connector 325 to measure a blood sugar value, which may be presented on the display 305.

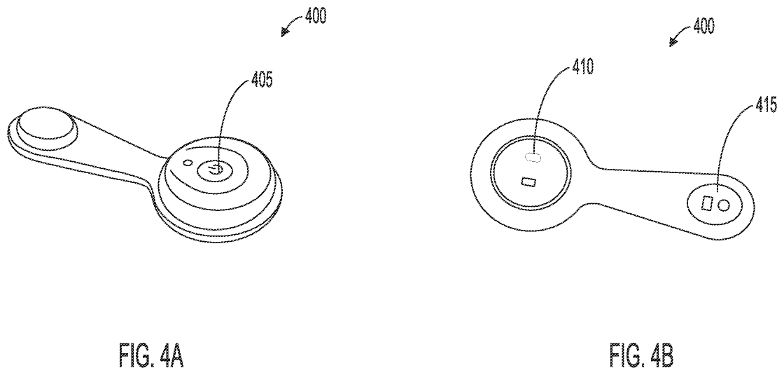

Referring to FIGS. 4A and 4B, illustrate example perspective views of an alternative example patch device 400 (e.g., another implementation of a wearable device 130) that may be utilized with the techniques described in connection with FIG. 1. As shown, the patch device 400 includes a power button 405, which may initiate the capture of sensor data points by the patch device 400. Like the card device 300 described in connection with FIGS. 3A-3C, the patch device 400 can include a portable power source, such as a battery (e.g., a lithium ion, alkaline, nickel metal hydride, etc.) or a device that can generate electric energy, such as a solar cell. As shown, the patch device 400 can include a primary set of sensors 410 and a secondary set of sensors 415. The primary set of sensors 410 and the secondary set of sensors 415 can include any of the sensors described herein, including PPG sensors, ECG sensors, UV sensors, motion sensors, temperature sensors, humidity sensors, pressure sensors, and blood sugar sensors, among others. The patch device 400 may be fixed to the user such that one or more of the sensors contact the skin of the user. The patch device 400 can be coupled to the user using an adhesive, a tape, or a mechanical fastener, among others. Like the card device 300 described in connection with FIGS. 3A-3C, the data points captured by the sensors of the patch device 400 can be communicated to other, external computing devices (not pictured) utilizing one or more communication interfaces, as described in further detail in connection with FIG. 1.

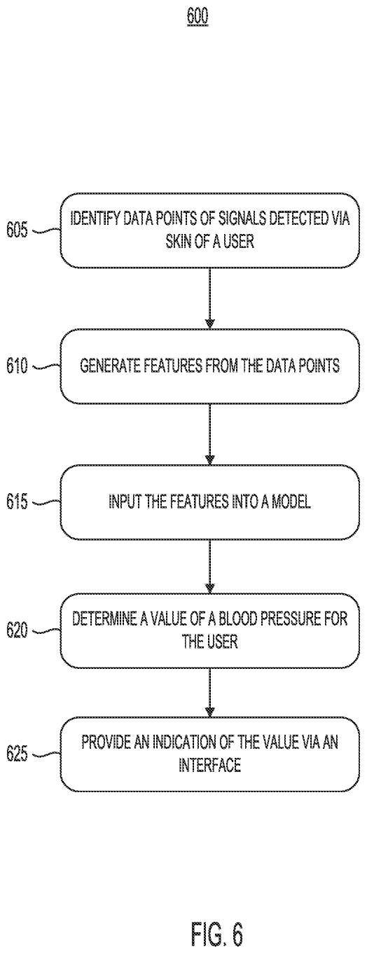

Referring now to FIG. 6, depicted is an illustrative flow diagram of a method 600 for monitoring a vital sign. The method 600 can be executed, performed, or otherwise carried out by the data processing system 105, the client device 120, the wearable device 130, or the computer system 900 described herein in connection with FIG. 9, or any other computing devices described herein. In brief overview, the method 600 can include identifying data points of signals detect via skin of a user at ACT 605. At ACT 610, the method 600 can include generating features from the data points. At ACT 615, the method 600 can include inputting the features into a model to produce output. At ACT 620, the method 600 can include determining a blood pressure value for the user. At ACT 625, the method 600 can include providing an indication of the blood pressure value.

Still referring to FIG. 6, and in further detail, at ACT 605, the method 600 can include identifying one or more data points (e.g., the data pointes 140) of signals detected via skin of a user by an optical sensor (e.g., one of the sensors 135 of the wearable device 130) that indicate changes in volume of blood flowing through a capillary at the skin. The data points may include PPG readings captured by an optical sensor of a wearable device (e.g., the wearable device 130). When represented as a time series, the data points can indicate changes in volume of blood flowing through a capillary at the skin of the user. The data points may be identified, for example, by transmitting a request for data points to a client device (e.g., the client device 120) or to the wearable device. In response to the request, the client device or the wearable device can transmit the raw data points to the data processing system, which may be time series data corresponding to a time period that the raw data points were captured. The data processing system can store the raw data points in association with an identifier of the patient from which the data points were captured, and in association with various metadata indicating, for example, the time period corresponding to the raw data points, the skin color of the user from which the data points were captured, or an identifier of the wearable device from which the raw data points were captured.

The data processing system can perform pre-processing on the raw data points captured by the sensors of the wearable device, for example, prior to performing one or more feature extraction processes. Pre-processing the raw data points can include performing at least one of a normalization technique, a detrending technique, or a smoothing technique. At a first stage of the pre-processing process, the data processing system can normalize the raw data points to the range from zero (0) to one (1), inclusive. Then, the data processing system can apply a detrending technique, which may include using a best straight-fit line from the normalized data points for a detrend function with default parameters. The data processing system can then apply a bandpass filter to the detrended data points. The bandpass filter may be any suitable type of bandpass filter, such as Chebyshev type II 4^{th} order bandpass filter. The bandpass filter may filter the detrended data points to a predetermined frequency range, such as 0.5 Hz to 10 Hz, and a predetermined attenuation, such as 20 db.

After applying the bandpass filter, the data processing system can apply further pre-processing operations. For example, the data processing system may apply a smoothing function. The smoothing function may be a three-point multi-pass moving average filter. The smoothing function may be applied for a predetermined number of iterations (e.g., three iterations). After applying the smoothing technique, the data processing system can then normalize the data points to the range from zero (0) to one (1), inclusive. Performing these pre-processing steps on the raw data points corresponding to PPG signals can accentuate different properties of interest in the PPG signals, such as the systolic peaks, diastolic peaks, and dicrotic notches.

After performing the aforementioned pre-processing techniques, the data processing system can perform a peak detection process to identify peaks in the pre-processed data points. These detected peaks can be used to extract segments of the data points that correspond to heart beats of the user. The heartbeat segments can then be used in a feature extraction process, as described in greater detail herein. The data processing system can apply a peak detection function to the pre-processed data points to identify one or more systolic peaks. The peak detection function may be, for example, an AMPD function. The peak detection function may be executed a predetermined number of times over the pre-processed data points (e.g., two passes, etc.). For example, a first pass of the AMPD function can be used to detect each peak in the pre-processed data points, and a second pass of the AMPD function can be used to detect each trough in the pre-processed data points.

Once the peaks and troughs have been identified in the pre-processed data points, the data processing system can generate one or more splices, or segments, of the pre-processed data points. These segments can be generated by splicing the pre-processed data points at each of the troughs. Once the segments are generated, the data processing system can calculate a time interval for each of the segments (e.g., by subtracting the timestamp of the first data point in the segment from the timestamp of the last data point in the segment). The data processing system can then calculate the mean time interval of all segments by summing each of the segment time intervals and dividing the sum by the number of segments.

The data processing system can use the mean time interval for the segments to determine which of the segments (which correspond to heartbeats) are valid. The valid segments can be utilized in further operations described herein, while the invalid segments can be discarded. To determine which of the segments are valid, the data processing system can compare the time interval of each of the segments to the mean time interval. Any segments having a time interval that is more than 1.5 times greater than the mean segment time interval can be considered invalid, and discarded. Additionally, the data processing system can discard any segments of the pre-processed data points that do not include a single detected peak between two detected troughs. After discarding the invalid segments, the data processing system is left with a set of valid segments of the pre-processed data points, which can be utilized in a feature extraction process.

At ACT 610, the method 600 can include generating one or more features (e.g., the extracted features) from the identified data points. The extracted features can include, for example, derivative values of the PPG signals in the valid segments. The derivative values can include first, second, third, and fourth order derivative values. Additionally, the extracted features can include heart rate variability, which can be calculated for each of the valid segments of the data points. To calculate a derivative, the data processing system can execute one or more discrete derivative functions, which can take the data points of a segment as input, and produce a corresponding set of derivative data points as output. The process may be repeated by utilizing the set of derivative data points as input to calculate a second derivative, and so on to calculate each of the first, second, third, and fourth derivative values. Each of these extracted features can be stored in association with the respective valid segment of the data points. In some implementations, the valid segment of the data points may itself be considered an extracted feature. The data processing system can also calculate the heart rate variability for each of the valid segments as an extracted feature, as well as the mean heart rate variability for all of the valid segments.

At ACT 615, the method 600 can include inputting the plurality of features into a model to produce output. The model may be trained using machine learning on training data having values for the plurality of features and labels corresponding to blood pressure measurements from a reference device different from the optical sensor. The output values can be estimates for the systolic and diastolic blood pressure of the user. The trained model can be a machine-learning model, such as an ensemble random forest model, and may be trained using any suitable supervised, unsupervised, or semi-supervised training model. The model can be trained prior to utilizing the model to estimate vital signs, by using sets of training data including ground truth data (e.g., labels). In some implementations, other types of models can be utilized, including linear regression models, SVM models, neural networks, or other types of regression models.

For example, the data processing system can train the model using training data that includes sets of extracted features corresponding to the features extracted by the data processing system, and also includes known values for the systolic and diastolic blood pressure. The sets of training data may correspond to a predetermined number of heartbeats monitored from a number of test subjects. At least three sets of data points can be gathered from each test subject under different conditions. A first set of the training data may gathered from test subjects in a resting, sitting position. A second set of the training data can be gathered from the test subjects in after performing a physical activity, such as jumping jacks. A third set of training data can be gathered from the test subjects after lying down. Each of the three sets of training data can be gathered a predetermined number of times for each test subject. For example, if the sets of training data are gathered for a test subject three times, then nine sets of training data are generated for that test subject.

To gather a set of training data from a test subject, the wearable device 130 can be used to generate raw data points corresponding to a predetermined time period (and therefore, a predetermined number of heartbeats). At the same time, systolic and diastolic blood pressure can be monitored and recorded for each heart beat using a cuff device. The raw data points for each beat can be provided to the data processing system and pre-processed and segmented, as described herein. After generating the segments, the data processing system can be used to generate the extracted features for each segment, as described herein. After generating the extracted features for each heartbeat, the known blood pressure values (e.g., the systolic and diastolic blood pressure) for each heartbeat can be stored in association with the corresponding set of extracted features, as the ground truth value. This generates one item of training data, which the data processing system can use to train the model. The process can be repeated for each heartbeat in each set of training data, to generate sets of training data for a test subject. In some implementations, the training data can be generated for several subjects, and the training data for the several subjects can be used to train a collective model.

To train the model, the data processing system can perform a machine learning function using the training data to adjust the parameters of the model. The machine learning function may be a random forest training function, which utilizes the known blood pressure values in each item of training data to compensate for the errors in the model. The trained model can be a regression model, which is trained using the machine learning function to output systolic and diastolic blood pressure values in response to receiving extracted features as input. The machine learning model may be an iterative training function, which trains the model using randomly selected batches of the training data until a predetermined accuracy level is reached.

To determine the accuracy of the model, a subset of the training data can be utilized as a test data set. The test data set can be a set of the training data that is not used to train the model, and is instead only used to evaluate the model accuracy. The accuracy can be evaluated by providing the extracted features in each item of the test data set as input to the model, and comparing the output of the model to the known values in the respective items of test data. The percentage of the output blood pressure values generated by the model that match the respective known blood pressure values in the test data (e.g., with a predetermined tolerance range) can be the overall accuracy of the model. The data processing system can repeatedly evaluate the accuracy of the model after training the model with a predetermined amount of training data. Once a predetermined accuracy value is reached, the data processing system can store the model in one or more data structures in the storage of the data processing system. The model can then be used to predict the blood pressure values (or values for other vital signals, if trained to do so) by using the extracted features.

In some implementations, the data processing system can generate and train a respective model for each user. For example, sets of training data can be gathered from a user, and a respective model can be trained for that user and later used to predict the blood pressure values (or other vital sign values) for that user. Likewise, in some implementations, a general model may be trained using training data from many users, and the general can be used to predict the blood pressure values (or other vital sign values) for many users, including users that did not necessarily contribute data for training. Once the model has been trained using the training data, the data processing system can provide the extracted features as input to the model, and execute the model to produce one or more output values.

At ACT 620, the method 600 can include determining, based on the output from the model, a value of blood pressure for the user. For example, the model may output the systolic and diastolic blood pressure for the user. The data processing system can store these values in association with an identifier of the user, indicating that the values represent the blood pressure of the user. In some implementations, the data processing system can store the output values in association with the respective extracted features provided as input to the model. In some implementations, the estimated blood pressure values can be determined based on an average of the blood pressure values generated by the model. For example, the prior operations described herein may generate several valid segments of data points that each represent a heartbeat, and sets of extracted features that correspond to each valid segment. The model can estimate blood pressure values for each set of features spanning a predetermined time window or a predetermined number of heartbeats, and average the values to determine a final blood pressure estimate. In some implementations, the data processing system can calculate a rolling average.

At ACT 625, the method 600 can include providing an indication of the value of blood pressure via an interface. For example, the data processing system can display the blood pressure values on a display of the data processing system. In some implementations, the data processing system can provide the blood pressure values for display on another computing device, such as a client device or the wearable device from which the data points were captured. The data processing system may transmit the calculated blood pressure values to the client device as they are calculated, such that they can be displayed to the user via a client application (e.g., the client application 125). The client application can transmit the blood pressure values to the wearable device, which can be presented on a display of the wearable device.

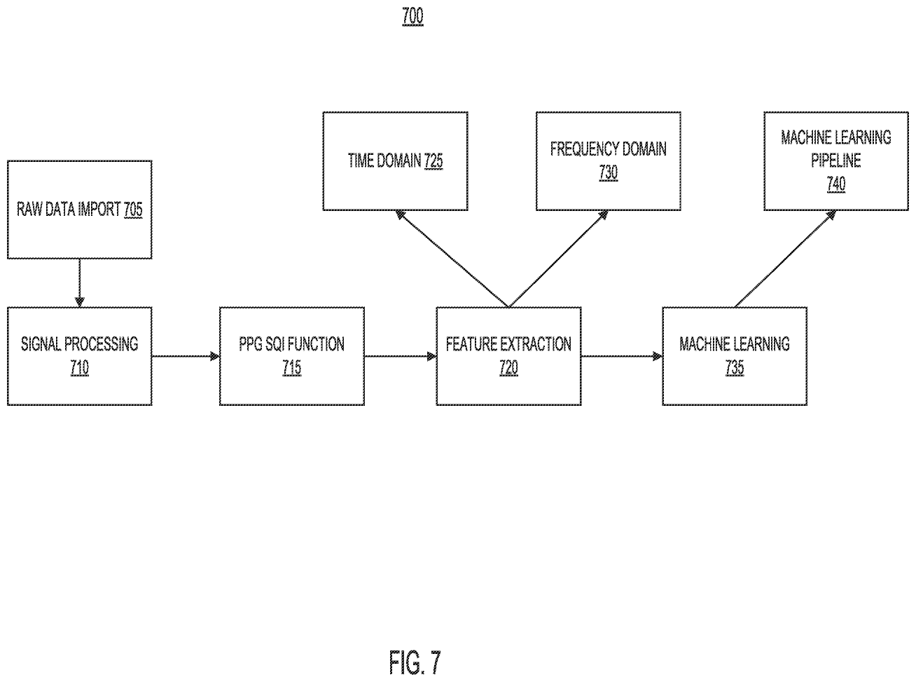

Referring to FIG. 7, illustrated is an example method 700 for monitoring vital signs via machine learning. The method 700 can be performed by one or more system or component depicted in FIGS. 1-5B or FIG. 9, including, for example, a data processing system or client device. The method 700 can include the data processing system importing raw data at ACT 705. The raw data can be collected by a wearable device or other client device. The raw data can include detections of optical signals. The optical signals can be detected in a predetermined frequency range or any electromagnetic frequency range. The optical signal can include infrared signals, green signals, red signals, or signals of other frequencies or wavelengths. In some cases, the optical sensors can be designed, constructed and operational to detect optical signals having certain characteristics. For example, the optical signals can detect signals having certain wavelengths and intensity, in which case the raw data can correspond to signals that satisfy the wavelength and intensity characteristics of the optical sensor.

The raw data can be collected or detected by a wearable device or client device. The raw data can be imported to a client application executing on the client device or wearable device. In some cases, the raw data can be imported to a data processing system or other processor or computing device communicatively coupled to the client device via a network. For example, the data processing system and wearable device can be located on a private network, such as a hospital network. The client device can establish a secure communication channel with the data processing system on the private network and transmit the raw data to the data processing system.

At ACT 710, the method 700 can include the data processing system performing signal processing. One or more processors of the client device or data processing system can perform signal processing on the raw data imported at ACT 705. The data processing system can perform various types of signal processing, including, for example, pre-processing techniques. The signal processing can include time domain pre-processing or frequency domain pre-processing.

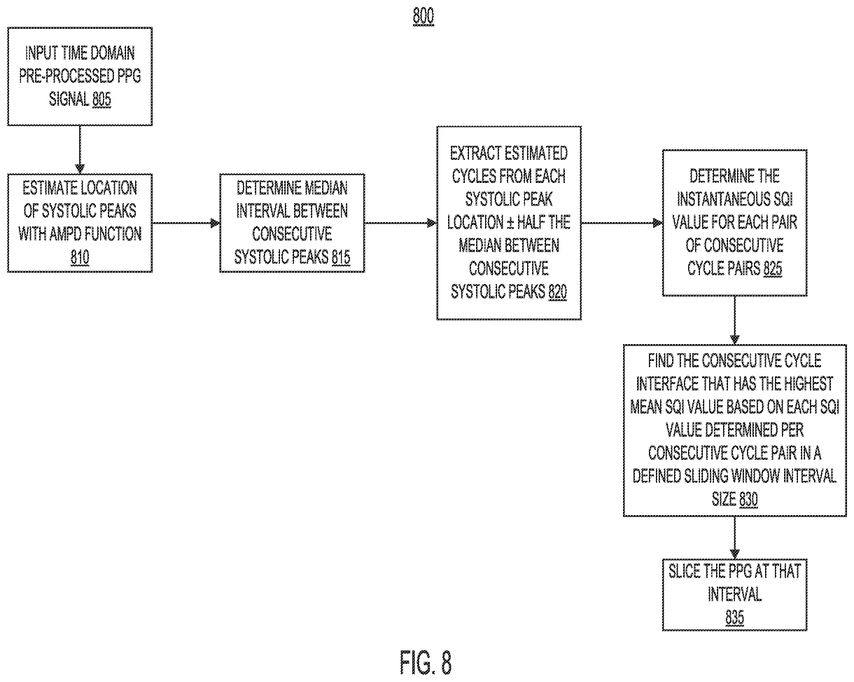

At ACT 715, the method 700 can include the data processing system executing a signal quality index (“SQI”) function. With the SQI function, the system can identify segments of the pre-processed data points that correspond to heartbeats. For example, the data processing system can identify a predetermined number (e.g., 5, 6, 7, 10, 12, or 15) of heartbeats that have the highest signal quality index. In some cases, the data processing system can apply a peak detection function to the data to identify one or more peaks, such as systolic peaks. Thus, using the SQI function, the data processing system can identify, generate or otherwise output PPG signals. The data processing system can execute the SQI function depicted in FIG. 8.

At ACT 720, the method 700 can include the data processing system extracting features. The data processing system can extract features by performing one or more operation on the PPG signals output from ACT 715. The data processing system can extract features in the time domain at ACT 725. The data processing system can extract features in the frequency domain at ACT 730. Obtaining features in the frequency domain can include or be based on obtaining the PPG signal, performing a Fourier transform (e.g., fast Fourier transform) of the PPG signal to transform the signal into the Fourier or frequency domain, and identifying features of the PPG in the frequency domain.

Extracting features in the time domain can include or be based on processing the PPG signal to identify peaks and troughs, isolating the valid PPG cycles, and then performing one or more derivatives on the valid PPG cycles. For example, the data processing system can take one or more derivatives of the PPG signal to generate features. For example, a first feature can be the PPG signal, a second feature can be a derivative of the PPG signal (e.g., a VPG signal), a third feature can be a second derivative of the PPG signal (e.g., an APG signal), a fourth feature can be a third derivative of the PPG signal, a fifth feature can be a fourth derivative of the PPG signal. The data processing system can generate, extract, or otherwise identify additional features. For example, a sixth feature can be an HRV signal. Features can include, for example, mean time domain features. In some cases, a feature can be based on or formed from a combination of features or signals. For example, a composite feature can be based on a combination of VPG and APG features. Another composite feature can be based on a combination of HRV and a composite feature. The data processing system can generate a statistic for each of the features and use the statistic for downstream processing. For example, the data processing system can generate an average or mean of the PPG features, VPG features, APG features, PPG3 features, or PPG4 features for each cycle time, and use the mean as the feature for input into the model.

At ACT 735, the method 700 can include the data processing system performing machine learning. Performing machine learning can refer to or include inputting the features into a model trained using machine learning so as to output a value or score indicative of a vital sign. For example, the data processing system can input the features extracted at ACT 720 into a model trained via machine learning to determine a value of a vital sign, such as a blood pressure reading.

In some cases, the data processing system can invoke a machine learning pipeline at ACT 740. The machine learning pipeline can include the data processing system importing data. The data processing system can obtain training data from a data repository. The training data can include the features recorded for a number of heartbeats (e.g., 120 beats) for each user in a group of users. The users can be grouped into different categories based on age, gender, demographics, geography, etc. The features can be generated for each user for a predetermined number of heart beats at each of a number of different levels of physical activity. For example, the training data can include, for each user, 120 beats worth of features when the user is sitting in a chair, 120 beats worth of features when after the user has performed a physical activity (e.g., walking, running, or jumping jacks), and 120 beats worth of data after the user has laid down for 2 minutes. The training data can include multiple (e.g., 3) sets of data for each user at each level of physical activity. For each set of 120 heart beats, the training data can include corresponding readings of the vital sign from a reference device, such as a mechanical-based cuff-based blood pressure monitor.

The machine learning pipeline can include the data processing system preparing the data. The machine learning pipeline can include the data processing system selecting features from the data. The machine learning pipeline can include the data processing system splitting the data into a training data set and a testing (e.g., hold out) data set. The data processing system can split the data such that 70% can be used for training, and 30% of the data can be used as a test or holdout data set, for example. The data processing system can build models with the training data set using a machine learning technique. The data processing system can use one or more machine learning techniques to train the model. For example, the data processing system can be configured with, include, or otherwise utilize a linear regression, logistic regression, decision tree, support-vector machine, k-nearest neighbor, k-means, or random forest.

The machine learning pipeline can include the data processing system testing the model built using the machine learning technique. The data processing system can test the model with the hold out data set. The data processing system can then evaluate the models based on the hold out data set. The data processing system can select one or more models to deploy based on the model satisfying the test or producing results for vital signs that match or are accurate within a threshold of the measurements from the reference device. The data processing system can save the satisfactory models in the data repository or in the model generator component of the data processing system. The data processing system can provide the selected models to the wearable device to monitor and display vital sign readings.

Referring to FIG. 8, illustrated is an example method 800 for generating signals used for monitoring vital signs. The method 800 can be performed by one or more system or component depicted in FIGS. 1-5B or FIG. 9, including, for example, a data processing system or client device. The method 800 can include the data processing system inputting time domain pre-processed PPG signals at ACT 805. The data processing system can receive the pre-processing PPG signals as an output of ACT 710 of method 700 depicted in FIG. 7.

At ACT 810, the method 800 can include estimating a location of systolic peaks with an peak detection function. The data processing system can be configured, include, or utilize one more peak detection function. For example, the data processing system can use an automatic multiscale-based peak detection (AMPD) function. The data processing system can execute the peak detection function one or more times, such as a predetermined number of times over the pre-processed PPG signal. For example, the data processing system can execute a first pass of the AMPD function to detect peaks in the pre-processed signals from ACT 805. The data processing system can execute a second pass of the AMPD function to detect troughs in the pre-processed PPG signals from ACT 805. By identifying the peaks and troughs using the peak detection function, the data processing system can determine, estimate, or otherwise identify a location of systolic peaks of the pre-processing PPG signal. The location can refer to a time stamp on an axis or a sample number on an axis in a graph, such as the sample value depicted in the graph in FIG. 5A.

At ACT 815, the method 800 can include the data processing system determining a median interval between consecutive systolic peaks. The data processing system can determine the median interval between the locations of the peaks identified at ACT 810. The data processing system can determine the median interval for a cycle, which can include a predetermined number of peaks or heartbeats (e.g., 120 beats worth of peaks). The data processing system can measure the median interval between peaks based on sample count or a time interval or other unit. For example, the data processing system can count the number of samples between consecutive systolic peaks.

At ACT 820, the data processing system can extract estimated cycles from a systolic peak location. The data processing system can extracted the estimated cycles form a systolic peak location plus or minus half the median between consecutive systolic peaks. By first identifying the median interval between consecutive peaks, the data processing system can determine a cycle which corresponds to a portion of the PPG signal. The cycle can be determined based on the median interval between consecutive peaks plus or minus a buffer or threshold, such as, for example, ½, ¼, ⅓, ⅕ of the median between consecutive peaks.

At ACT 825, the data processing system can determine an instantaneous signal quality value (“SQI”) value for each pair of consecutive cycle pairs. The SQI value can refer to or indicate the reliability with which the peaks in the one or more cycles capture a metric or feature of one or more heartbeats in the one or more cycles. The SQI value can be a numeric value that indicates a level of quality of the signal. For example, the SQI value can range from 1 to 5, where 1 indicates very low quality and 5 indicates very high quality. The SQI value can have any other range, or type of value. The data processing system can use any type of SQI to determine the quality of the signals. Example metrics used to determine SQI can include perfusion, kurtosis, skewness, relative power, non-stationarity, zero crossing, entropy, or the matching of systolic wave detectors. The data processing system can use one or more metrics to generate an SQI value. The data processing system can combine multiple metrics to generate an SQI value.

At ACT 830, the data processing system can identify a consecutive cycle interface with a highest mean SQI value. The data processing system can identify the highest SQI value based on each SQI value determined at ACT 825 per consecutive cycle pair in a defined sliding window interval size. The data processing system can define the sliding window interval size based on the median between consecutive peaks determined at ACT 820.

At ACT 835, the data processing system can slice the PPG signal at the interval corresponding to the consecutive cycle interface with the highest mean SQI value. Slicing the PPG signal can refer to or include combining, connecting, joining or otherwise putting together PPG signals that the data processing system determines to be of high quality. The data processing system can input the spliced PPG signals at ACT 720 depicted in FIG. 7 to perform feature extraction. By performing the method 800 to generate high quality PPG signals for feature extraction the data processing system can reduce erroneous readings of vital signs and improve the accuracy of determining vital signs using the machine learning model. For example, the data processing system can improve the quality of the data that is input into the machine learning model and used to generate a value for a vital sign. By improving the quality using the SQI function, for example, the data processing system can improve the reliability and accuracy of determining vital signs from optical signals using a model trained via machine learning.

Referring now to FIG. 9, depicted is a block diagram of an example computer system 900. The computer system or computing device 900 can include or be used to implement portions of the data processing system 105, the client device 120, or the wearable device 130 described in connection with FIG. 1. The computing system 900 includes at least one bus 905 or other communication component for communicating information and at least one processor 910 or processing circuit coupled to the bus 905 for processing information. The computing system 900 can also include one or more processors 910 or processing circuits coupled to the bus 905 for processing information. The computing system 900 also includes at least one main memory 915, such as a RAM or other dynamic storage device, coupled to the bus 905 for storing information, and instructions to be executed by the processor 910. The computing system 900 may further include at least one ROM 920 or other static storage device coupled to the bus 905 for storing static information and instructions for the processor 910. A storage device 925, such as a solid state device, magnetic disk, or optical disk, can be coupled to the bus 905 to persistently store information and instructions.

The computing system 900 may be coupled via the bus 905 to a display 935, such as a liquid crystal display, or active matrix display, for displaying information to a user such as an administrator of the data processing system. An input device 930, such as a keyboard or voice interface may be coupled to the bus 905 for communicating information and commands to the processor 910. The input device 930 can include a touch screen display 935. The input device 930 can also include a cursor control, such as a mouse, a trackball, or cursor direction keys, for communicating direction information and command selections to the processor 910 and for controlling cursor movement on the display 935. The display 935 can be part of, or communicatively coupled to, the wearable device 130 or client device 120, or other components of FIG. 1.

The processes, systems, and methods described herein can be implemented by the computing system 900 in response to the processor 910 executing an arrangement of instructions contained in main memory 915. Such instructions can be read into main memory 915 from another computer-readable medium, such as the storage device 925. Execution of the arrangement of instructions contained in main memory 915 causes the computing system 900 to perform the illustrative processes described herein. One or more processors in a multi-processing arrangement can be employed to execute the instructions contained in main memory 915. Hard-wired circuitry can be used in place of or in combination with software instructions together with the systems and methods described herein. Systems and methods described herein are not limited to any specific combination of hardware circuitry and software.

Although an example computing system has been described in FIG. 9, the subject matter including the operations described in this specification can be implemented in other types of digital electronic circuitry, or in computer software, firmware, or hardware, including the structures disclosed in this specification and their structural equivalents, or in combinations of one or more of them.

Implementations of the subject matter and the operations described in this specification can be implemented in digital electronic circuitry, or in computer software embodied on a tangible medium, firmware, or hardware, including the structures disclosed in this specification and their structural equivalents, or in combinations of one or more of them.

Implementations of the subject matter described in this specification can be implemented as one or more computer programs, e.g., one or more components of computer program instructions, encoded on computer storage medium for execution by, or to control the operation of, data processing apparatus. The program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can include a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The terms “data processing apparatus”, “data processing system”, “client device”, “computing platform”, “computing device”, or “device” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA or an ASIC.

Processors suitable for the execution of a computer program include, by way of example, both general and special purpose microprocessors, and any one or more processors of any kind of digital computer. Generally, a processor will receive instructions and data from a read-only memory or a random access memory or both. The elements of a computer include a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), for example. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube), plasma, or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can include any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system such as the data processing system 105 may include clients and servers. For example, the data processing system 105 can include one or more servers in one or more data centers or server farms. A client and server can be remote from each other and can interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving input from a user interacting with the client device). Data generated at the client device (e.g., a result of an interaction, computation, or any other event or computation) can be received from the client device at the server, and vice-versa.

While this specification contains many specific implementation details, these should not be construed as limitations on the scope of any disclosure or of what may be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. For example, the data processing system 105 could be a single module, a logic device having one or more processing modules, or one or more servers.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed only in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. Although the examples provided may be useful for monitoring a vital sign, the systems and methods described herein may be applied to other environments. The foregoing implementations are illustrative rather than limiting of the described systems and methods. The scope of the systems and methods described herein may thus be indicated by the appended claims, rather than the foregoing description, and changes that come within the meaning and range of equivalency of the claims are embraced therein.

What is claimed is:

1. A system to monitor a vital sign, comprising:
a data processing system comprising one or more processors, coupled to memory, to:
identify a plurality of data points of signals detected via skin of a user by an optical sensor that indicate changes in volume of blood flowing through a capillary at the skin;
generate a plurality of features from the plurality of data points;
input the plurality of features into a model to produce output, the model trained using machine learning on training data having values for the plurality of features and labels corresponding to blood pressure measurements from a reference device different from the optical sensor;
determine, based on the output from the model, a value of blood pressure for the user; and
provide an indication of the value of blood pressure via an interface.

2. The system of claim 1, wherein the signals are photoplethysmogram (“PPG”) signals obtained from the optical sensor.

3. The system of claim 1, comprising:
the data processing system to execute a derivative of the plurality of data points to generate a first feature of the plurality of features.

4. The system of claim 3, comprising the data processing system to:
execute a second derivative of the plurality of data points to generate a second feature of the signals; and
update the value of the blood pressure based on the first feature and the second feature input into the model.

5. The system of claim 4, wherein a third feature of the plurality of features comprises heart rate variability.

6. The system of claim 1, comprising:
the data processing system to pre-process the signals detected by the optical sensor to generate the plurality of data points using at least one of a normalization technique, detrending technique, or a smoothing technique.

7. The system of claim 1, comprising:
the data processing system to filter the signals based on a frequency range to generate the plurality of data points.

8. The system of claim 1, comprising:
the data processing system to apply a peak detection technique to the signals to generate the plurality of data points.

9. The system of claim 1, comprising the data processing system to:
identify a plurality of peaks in the signals and a plurality of troughs in the signals;
generate a plurality of splices of the signals based on the plurality of troughs;
discard one or more of the plurality of splices having a duration greater than a threshold; and
generate the plurality of data points absent the one or more of the plurality of splices discarded responsive to the duration of the one or more of the plurality of splices being greater than the threshold.

10. The system of claim 1, wherein the machine learning comprises a random forest machine learning technique.

11. The system of claim 1, comprising:
the data processing system to receive, via a network from a computing device worn by the user, the signals, wherein the computing device comprises the optical sensor that detects the signals via the skin of the user.

12. The system of claim 1, comprising:
a computing device worn by the user, wherein the computing device comprises:
the data processing system; and
the interface comprises a display to provide the indication of the value of blood pressure.

13. The system of claim 1, comprising the data processing system to:
receive the training data comprising, for each of a plurality of users:
values for the plurality of features generated from a predetermined number of data points of signals corresponding to a predetermined number of heart beats of the plurality of users;
blood pressure observations measured by the reference device for each of the predetermined number of heart beats, wherein the reference device is different from the optical sensor.

14. The system of claim 13, wherein a first set of the training data corresponds to the plurality of users performing a first level of physical activity, a second set of the training data corresponds to the plurality of users performing a second level of physical activity different from the first level of physical activity, and a third set of the training data corresponds to the plurality of users performing a third level of physical activity that is different from the first level of physical activity and the second level of physical activity.

15. The system of claim 1, comprising the data processing system to:
detect, via the optical sensor, a color of the skin of the user; and
adjust an intensity of a frequency of light emitted based on the color of the skin to reduce erroneous data points of the plurality of data points.

16. A method of monitoring a vital sign, comprising:
identifying, by a data processing system comprising one or more processors coupled to memory, a plurality of data points of signals detected via skin of a user by an optical sensor that indicate changes in volume of blood flowing through a capillary at the skin;
generating, by the data processing system, a plurality of features from the plurality of data points;
inputting, by the data processing system, the plurality of features into a model to produce output, the model trained using machine learning on training data having values for the plurality of features and labels corresponding to measurements of the vital sign from a reference device different from the optical sensor;
determining, by the data processing system based on the output from the model, a value of the vital sign for the user; and
providing, by the data processing system, an indication of the value of the vital sign via an interface.

17. The method of claim 16, wherein the signals are photoplethysmogram (“PPG”) signals obtained from the optical sensor.

18. The method of claim 16, comprising:
executing, by the data processing system, a derivative of the plurality of data points to generate a first feature of the plurality of features.

19. An apparatus wearable by a user to monitor a vital sign, comprising:
an optical sensor;
a display;
a data processing system comprising one or more processors, coupled to memory, to:
identify a plurality of data points of signals detected via skin of the user by the optical sensor that indicate changes in volume of blood flowing through a capillary at the skin;
generate a plurality of features from the plurality of data points;
input the plurality of features into a model to produce output, the model trained using machine learning on training data having values for the plurality of features and labels corresponding to blood pressure measurements from a reference device different from the optical sensor;
determine, based on the output from the model, a value of blood pressure for the user; and
provide an indication of the blood pressure via the display.

20. The apparatus of claim 19, comprising:
the data processing system to pre-process the signals detected by the optical sensor to generate the plurality of data points using at least one of a normalization technique, detrending technique, or a smoothing technique.
